(19) United States
(12) Patent Application Publication
(10) Pub. No.: US 2014/0044780 A1
(43) Pub. Date: Feb. 13, 2014

(54) Extended-Release Levetiracetam and Method of Preparation

(71) Applicant: PharmTak, Inc., San Jose, CA (US)

(72) Inventors: I-Lan Tung Sue, San Jose, CA (US); Jung-Chung Lee, Saratoga, CA (US)

(21) Appl. No.: 13/958,933

(22) Filed: Aug. 5, 2013

Related U.S. Application Data
(60) Provisional application No. 61/680,789, filed on Aug. 8, 2012.

(51) Int. Cl. A61K 9/14; A61K 31/4015

(52) U.S. Cl. CPC A61K 9/146; A61K 31/4015
USPC 424/458; 424/465; 424/488; 424/490; 424/494; 514/424

(57) ABSTRACT

Described herein is a pharmaceutical composition comprising levetiracetam as an active ingredient to provide prolonged release characteristic to allow once a day dosage regime. The innovative formulation comprises levetiracetam and a hydrophobic polymer with or without additional release rate modifier(s). The formulation may comprise other pharmaceutically acceptable excipients. This invention also describes the processes of preparing such dosage forms.

### CROSS-REFERENCE TO RELATED APPLICATIONS

This application claims the benefit of U.S. Provisional Application No. 61/680,789, filed on Aug. 8, 2012, which is incorporated by reference herein in its entirety.

### TECHNICAL FIELD

This invention relates to an extended release dosage form(s) of levetiracetam including the pharmaceutical composition and methods for producing them.

### BACKGROUND

The chemical name of levetiracetam, a single enantiomer, is (−)-(S)-α-ethyl-2-oxo-1-pyrrolidine acetamide, its molecular formula is C₈H₁₄N₂O₂ and its molecular weight is 170.21. Levetiracetam is used as an antiepileptic drug (AED). It has the following structural formula:

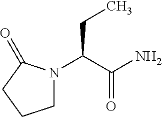

Levetiracetam is very soluble in water (104 g/100 mL). It is challenging to develop a dosage form to control or delay levetiracetam release from the dosage form when placed in water or consumed by oral administration.

U.S. Pat. No. 7,858,122 uses a hydrophilic matrix to produce levetiracetam tablets which can be administered orally, permitting controlled release with a once a day dosage regime. U.S. Pat. No. 7,863,316 comprises a core tablet with a water dispersible rate controlling polymer and a functional coating. The amount of water used in these processes is minimal due to levetiracetam's high solubility in water.

Levetiracetam, being a highly soluble and high dose drug substance, poses a challenge to the formulator for developing controlled release rate dosage forms. When large quantities of excipients are used to control the release, dosage form sizes will be large, making oral administration problematic.

There remains a need, however, for improved oral pharmaceutical formulations for the extended-release of active agents, e.g., levetiracetam, to allow for reduced frequency of administration. Also needed are dosage formulations having substantially no food effect such that a patient has the convenience of taking the dosage form with or without food.

### BRIEF SUMMARY OF THE INVENTION

Described herein is a newly developed method of producing an extended release formulation comprising a reservoir particulate with a levetiracetam core and a hydrophobic polymer coating. Using this newly developed approach, an extended release levetiracetam dosage form that exhibits controlled or extended release of levetiracetam in either in vitro or in vivo conditions is provided.

In a first aspect there is provided a reservoir particulate comprising a levetiracetam core coated with an aqueous dispersion of a hydrophobic polymer. In an embodiment, the aqueous dispersion contains no organic solvents. In another embodiment, the aqueous dispersion is substantially free of organic solvents.

In another embodiment the levetiracetam core comprises levetiracetam, a pharmaceutically acceptable salt, solvate, hydrate, crystalline form, non-crystalline form or combination thereof.

In one embodiment, the reservoir particulate comprises levetiracetam at a concentration of about 50% w/w to about 95% w/w. In another embodiment, the reservoir particulate comprises levetiracetam at a concentration of about 60% w/w to about 90% w/w. In another embodiment, the reservoir particulate comprises levetiracetam at a concentration of about 65% w/w to about 80% w/w. In another embodiment, the reservoir particulate comprises levetiracetam at a concentration of about 73% w/w to about 79% w/w.

In another embodiment the hydrophobic polymer is selected from ethyl cellulose, cellulose acetate, polyvinyl acetate, methacrylic acid esters neutral polymer, polyvinyl alcohol-maleic anhydride copolymers and combinations thereof, preferably ethyl cellulose. In a further embodiment, the hydrophobic polymer is present at a concentration of about 3-30% w/w in the aqueous dispersion prior to use. In another embodiment, the hydrophobic polymer is present at a concentration of about 3-40% w/w in the aqueous dispersion prior to use. In another embodiment, the aqueous dispersion further comprises at least one excipient selected from a plasticizer, a suspending agent, an anti-caking agent, an emulsifying agent, and an anti-coagulation agent.

In one embodiment, the reservoir particulate comprises hydrophobic polymer(s) at a concentration of about 5% w/w to about 50% w/w. In another embodiment, the reservoir particulate comprises hydrophobic polymer(s) at a concentration of about 10% w/w to about 30% w/w. In another embodiment, the reservoir particulate comprises hydrophobic polymer(s) at a concentration of about 10% w/w to about 20% w/w.

In an embodiment, the aqueous dispersion further comprises a hydrophilic polymer. In a further embodiment, the hydrophilic polymer is selected from copolyvidone, polyvinyl pyrrolidone, polyethylene glycols, hydroxyethyl cellulose, hydroxypropyl methylcellulose, hydroxypropyl cellulose and combinations thereof. In specific embodiments, the hydrophilic polymer is hydroxypropyl methylcellulose. In a yet further embodiment, the hydrophilic polymer(s) are present at a concentration of about 0.5-10% w/w in the aqueous dispersion prior to use. In a further embodiment, the hydrophilic excipient is selected from polyvinyl alcohol (PVA), agar, sodium alginate, alginic acid and gelatin.

In one embodiment, the reservoir particulate comprises hydrophilic polymer(s) at a concentration of 0% to about 19% w/w (e.g., up to about 19% w/w). In another embodiment, the reservoir particulate comprises hydrophilic polymer(s) at a concentration of 0% to about 5% w/w (e.g., up to about 5% w/w). In another embodiment, the reservoir particulate comprises hydrophilic polymer(s) at a concentration of about 3% w/w to about 15% w/w. In another embodiment, the reservoir particulate comprises hydrophilic polymer(s) at a concentration of about 3% w/w to about 13% w/w. In another embodiment, the reservoir particulate comprises hydrophilic polymer(s) at a concentration of about 3% w/w to about 5% w/w.

In another embodiment, the reservoir particulate comprises levetiracetam at an amount from about 50% to 95% w/w, the hydrophobic polymer is about 5% to 50% w/w, and the hydrophilic polymers are about 0% to 19% w/w of the particulate.

In another embodiment, the reservoir particulate comprises levetiracetam at a concentration of about 65% w/w to about 80% w/w, a hydrophobic polymer at a concentration of about 10% w/w to about 30% w/w, and a hydrophilic polymer at a concentration of about 3% w/w to about 13% w/w.

In another embodiment, the reservoir particulate comprises levetiracetam at a concentration of about 73% w/w to about 79% w/w, a hydrophobic polymer at a concentration of about 10% w/w to about 20% w/w, and a hydrophilic polymer at a concentration of about 3% w/w to about 5% w/w.

In some embodiments, the hydrophobic polymer is ethyl cellulose and the hydrophilic polymer is hydroxypropyl methylcellulose.

In an embodiment, there is a reservoir particulate comprising:
a) Levetiracetam or a pharmaceutically acceptable salt, solvate, hydrate, crystalline form, non-crystalline form or combination thereof;
b) at least one hydrophobic polymer; and
c) at least one hydrophilic polymer,
   wherein the polymers are in an aqueous dispersion that is coated onto the levetiracetam during granulation. In an embodiment, the aqueous dispersion is free of or substantially free of organic solvents. In an embodiment, the hydrophobic polymer and the hydrophilic polymer form a coating on the levetiracetam.

In an embodiment, the reservoir particulate comprises particles and/or agglomerates.

In another embodiment, the hydrophilic polymer is mixed with the hydrophobic polymer prior to addition to the levetiracetam. In a further embodiment, the coating is partial.

In a yet further embodiment, the hydrophobic polymer, when compressed into tablet dosage form, forms a cross-linked structure (matrix) which will not dissolve in water. The hydrophobic polymer provides a mechanism in controlling levetiracetam release from the reservoir particulate. In an embodiment, the hydrophobic polymer maintains the original shape of the dosage form for up to at least 12 hours in vitro. The dosage form is a tablet, mini-tab, pellet, bead or pill and the like.

In an embodiment, the reservoir particulate comprises levetiracetam in an amount of about from 50% to 95% w/w, the hydrophobic polymer is about 5% to 50% w/w, and the hydrophilic polymers are about 0.1% to 19% w/w of the particulate. In an embodiment, the reservoir particulate comprises levetiracetam in an amount of about 50% to 95% w/w, the hydrophobic polymer is about 5% to 50% w/w, and the hydrophilic polymers are about 0.1% to 30% in total.

In an embodiment, the hydrophobic polymer is selected from ethyl cellulose, cellulose acetate, polyvinyl acetate, methacrylic acid esters neutral polymer, polyvinyl alcohol-maleic anhydride copolymers and combinations thereof. In a further embodiment, the hydrophobic polymer is ethyl cellulose. In a yet further embodiment, the hydrophobic polymer is present at a concentration of about 3-30% w/w in the aqueous dispersion prior to use. In an embodiment, the hydrophobic polymer is present at a concentration of about 3-40% w/w in the aqueous dispersion prior to use. In another embodiment, the aqueous dispersion further comprises at least one excipient selected from a plasticizer, a suspending agent, an anti-caking agent, an emulsifying agent, and an anti-coagulation agent.

In an embodiment, the hydrophilic polymer is selected from copolyvidone, polyvinyl pyrrolidone, polyethylene glycols, hydroxyethyl cellulose, hydroxypropyl methylcellulose, hydroxypropyl cellulose and combinations thereof. In specific embodiments, the hydrophilic polymer is hydroxypropyl methylcellulose. In certain embodiments, the hydrophilic polymer(s) are present at a concentration of about 0.5-10% w/w in the aqueous dispersion prior to use.

In a second aspect there is a method of manufacturing an extended-release pharmaceutical composition, said method comprising:
a) coating levetiracetam with an aqueous dispersion of a hydrophobic polymer to form a reservoir particulate.

In an embodiment, the method comprises:
a) coating levetiracetam with an aqueous dispersion of a hydrophobic polymer to form a reservoir particulate;
b) blending the reservoir particulates of step (a) with one or more pharmaceutically acceptable excipients to form a dry blend; and
c) forming a final dosage form from the dry blend.

In another embodiment, the method further comprises:
d) coating the dosage form with a controlled release layer.

In one embodiment, the aqueous dispersion further comprises a hydrophilic polymer.

In one embodiment, the coating is done in a spray dry granulator, a high shear granulator or a fluid bed granulator. The fluid bed granulator is selected from a top spray, bottom spray or side (i.e., tangential) spray granulator.

In an embodiment, the pharmaceutically acceptable excipient is selected from the group comprising a hydrophilic polymer, binders, lubricants, glidants, disintegrants, fillers, diluents and combinations thereof. The hydrophilic polymer may be the same as or different from the hydrophilic polymer from the one used in the reservoir particulate, if one is in the reservoir particulate. The hydrophilic polymer is a dry powder. In certain embodiments, the hydrophilic polymer is hydroxypropyl methylcellulose.

In a further embodiment, the method further comprises sizing the reservoir particulate of step (a) (e.g., prior to step (b)). In some embodiments, the reservoir particle has a particle size of from about 10 μm to about 1000 μm. In some embodiments, the reservoir particle has a median particle size of from about 200 μm to about 700 μm or about 100 μm to about 700 μm, preferably about 500 μm. In various embodiments, the reservoir particle has a median particle size of any of about 10, 50, 100, 150, 200, 250, 300, 350, 400, 450, 500, 550, 600, 650, 700, 750, 800, 850, 900, 950, or 1000 μm.

In some embodiments, the moisture content of the reservoir particles is about 0.05-5% w/w, preferably the reservoir particles are dried to less than 1.0% w/w water content as measured by weight loss using the loss on drying (LOD) method at 105° C.

In embodiments, the final dosage form is a tablet, capsule, sachet, mini-tabs, pellets or free flowing granules. In an embodiment, a tablet comprising a reservoir particulate as described herein is provided. In an embodiment, a capsule or sachet comprising a reservoir particulate as described herein is provided.

In a third aspect there is an extended-release pharmaceutical composition prepared by a method comprising:
a) coating levetiracetam with an aqueous dispersion of a hydrophobic polymer to form a reservoir particulate.

In one embodiment, the method comprises:
a) coating levetiracetam with an aqueous dispersion of a hydrophobic polymer to form a reservoir particulate;
b) blending the reservoir particulates of step (a) with one or more pharmaceutically acceptable excipients to form a dry blend; and
c) forming a final dosage form from the dry blend.

In a further embodiment, the method further comprises:
d) coating the dosage form with a controlled release layer.

In an embodiment, the extended-release pharmaceutical composition further comprises a controlled release layer. The controlled release layer comprises at least one hydrophobic excipient. The hydrophobic excipient is a hydrophobic polymer.

In another embodiment, the controlled release layer comprises at least one hydrophilic excipient. The hydrophilic excipient is selected from a hydrophilic polymer, propylene glycol, sucrose, xylitol and sodium lauryl sulfate. In specific embodiments, the hydrophilic excipient is a hydrophilic polymer.

In a further embodiment, the controlled release layer comprises at least one hydrophilic excipient and one hydrophobic excipient.

In a fourth aspect there is an extended-release pharmaceutical composition comprising
a) a reservoir particulate, wherein said particulate has a levetiracetam core coated with an aqueous dispersion of a hydrophobic polymer.

In an embodiment, the composition comprises:
a) a reservoir particulate, wherein said particulate has a levetiracetam core coated with an aqueous dispersion of a hydrophobic polymer, and
b) an extra-particulate matrix, wherein said matrix comprises a pharmaceutically acceptable excipient.

In an embodiment, the composition comprises:
a) a reservoir particulate, wherein said particulate has a levetiracetam core coated with an aqueous dispersion of a hydrophobic polymer;
b) an extra-particulate matrix, wherein said matrix comprises at least one hydrophilic polymer.

In an embodiment of the extended-release pharmaceutical composition, the levetiracetam core is levetiracetam or a pharmaceutically acceptable salt, solvate, hydrate, crystalline form or non-crystalline form thereof.

In an embodiment of the extended-release pharmaceutical composition, the aqueous dispersion of the hydrophobic polymer further comprises a hydrophilic polymer.

In some embodiments, both the reservoir particulate and the extra-particulate matrix comprise at least one hydrophilic polymer, wherien the hydrophilic polymer(s) in the reservoir particulate and the extra-particulate matrix are the same or different. In one embodiment, the hydrophilic polymer in both the reservoir particulate and the extra-particulate matrix is hydroxypropyl methylcellulose.

In an embodiment of the extended-release pharmaceutical composition, the composition comprises:
a) a reservoir particulate, wherein said particulate has a levetiracetam core coated with an aqueous dispersion of ethyl cellulose and hydroxypropyl methylcellulose;
b) an extra-particulate matrix, wherein said matrix comprises hydroxypropyl methylcellulose, lactose, colloidal silicon dioxide and magnesium stearate.

In an embodiment of the extended-release pharmaceutical compostion, the composition comprises:
a) a reservoir particualte, wherien said particulate has a levetiracetam core coaed with an aqueous dispersion of ethyl cellulose and hydroxypropyl methylcellulose;
b) an extra-particualte matrix, wherein said matrix comprises at least one pharmaceutically acceptable excipient selected from hydroxypropyl methylcellulose, lactose, colloidal silicon dioxide, magnesium stearate, and combinations thereof.

In another embodiment of the extended-release pharmaceutical composition, the composition comprises:
a) a reservoir particulate, wherein said particulate has a levetiracetam core coated with an aqueous dispersion of ethyl cellulose and hydroxypropyl methylcellulose;
b) an extra-particulate matrix, wherein said matrix comprises hydroxypropyl methylcellulose, colloidal silicon dioxide and magnesium stearate.

In another embodiment of the extended-release pharmaceutical composition, the composition comprises:
a) a reservoir particulate, wherein said particulate has a levetiracetam core coated with an aqueous dispersion of ethyl cellulose and hydroxypropyl methylcellulose;
b) an extra-particulate matrix, wherein said matrix comprises hydroxypropyl methylcellulose and magnesium stearate.

In one embodiment of the extended-release pharmaceutical composition, the levetiracetam is present at a concentration of about 30% w/w to about 95% w/w, and the hydrophobic polymer is present at a concentration of about 2% w/w to about 50% w/w.

In another embodiment of the extended-release pharmaceutical composition, the levetiracetam is present at a concentration of about 30% w/w to about 95% w/w, the hydrophobic polymer is present at a concentration of about 2% w/w to about 50% w/w, and at least one hydrophilic polymer is present at a concentration up to about 19% w/w.

In another embodiment of the extended-release pharmaceutical composition, the levetiracetam is present at a concentration of about 50% w/w to about 90% w/w, the hydrophobic polymer is present at a concentration of about 5% w/w to about 30% w/w, and at least one hydrophilic polymer is present at a concentration of about 5% w/w to about 19% w/w.

In another embodiment of the extended-release pharmaceutical composition, the levetiracetam is present at a concentration of about 72% w/w, a hydrophobic polymer is present at a concentration of about 12% w/w, and a hydrophilic polymer is present at a concentration of about 10% w/w, formlated in a tablet that comprises about 750 mg levetiracetam.

In another embodiment of the extended-release pharmaceutical composition, the levetiracetam is present at a concentration of about 70% w/w, a hydrophobic polymer is present at a concentration of about 12% w/w, and a hydrophilic polymer is present at a concentration of about 11% w/w, formlated in a tablet that comprises about 500 mg levetiracetam

In a fifth aspect there is an extended-release pharmaceutical composition, comprising a matrix comprising at least one hydrophilic material, one glidant, one diluent and one lubricant, and at least one reservoir particulate comprising levetiracetam or a derivative thereof (e.g., levetiracetam or a pharmaceutically acceptable salt, solvate, hydrate, crystalline form or non-crystalline form thereof) coated with at least one hydrophobic polymer, one hydrophilic polymer, or a combination thereof in an aqueous dispersion, wherein the composition is free of organic solvents. In one embodiment, the at least one reservoir particulate is a plurality of reservoir particulates.

In a sixth aspect there is an extended-release pharmaceutical composition, comprising a matrix comprising at least one hydrophilic material, one glidant, one diluent and one lubricant, and at least one reservoir particulate comprising levetiracetam or a derivative thereof (e.g., levetiracetam or a pharmaceutically acceptable salt, solvate, hydrate, crystalline form or non-crystalline form thereof) coated with at least one hydrophobic polymer, one hydrophilic polymer, or a combination thereof in an aqueous dispersion, wherein the composition contains no substantial amount of organic solvents. In certain embodiments, the extended-release pharmaceutical composition is produced without the addition of organic solvents. In one embodiment, the at least one reservoir particulate is a plurality of reservoir particulates.

In a seventh aspect there is an extended-release pharmaceutical composition, comprising reservoir particulates consisting of levetiracetam, a pharmaceutically acceptable salt, solvate, hydrate, crystalline form or non-crystalline form thereof, and a hydrophobic polymer, wherein the composition is free of organic solvents.

In some embodiments the extended-release pharmaceutical composition is intended to be administered once daily. In some embodiments, the extended-release pharmaceutical composition is intended to be administered orally once daily.

In some embodiments of the extended-release pharmaceutical composition(s) provided for herein, the hydrophobic polymer is present in an amount of about 2% to 50% w/w per total weight of the pharmaceutical composition. The hydrophobic polymer is preferably ethylcellulose.

In some embodiments of the extended-release pharmaceutical composition(s) provided for herein, the hydrophilic polymer is present in an amount of about 0% to about 38% w/w per total weight of the pharmaceutical composition (e.g., the hydrophilic polymer is present in an amount of about 0% w/w to about 19% w/w in each of the coat and/or matrix. The hydrophilic polymer is preferably hydroxypropyl methylcellulose.

In an eighth aspect, there is an extended-release pharmaceutical composition, wherein the pharmaceutical composition releases the levetiracetam contained therein over a period of about 12 hours after introduction of the dosage form into the dissolution medium when tested in 900 mL of pH 6.0 phosphate buffer maintained at 37° C. using a basket method (USP Apparatus 1) at 100 rpm. In one embodiment, the pharmaceutical composition releases about 85 wt. % to about 100 wt. % of the levetiracetam contained therein over a period of about 12 hours after introduction of the dosage form into the dissolution medium when tested in 900 mL of pH 6.0 phosphate buffer maintained at 37° C. using a basket method (USP Apparatus 1) at 100 rpm.

In a ninth aspect there is a once-daily oral extended-release pharmaceutical composition in a unit dosage form wherein the release rate of levetiracetam is on an average in the range from about 3% to about 9% per hour over a 12 hour period. In one embodiment, the rate of release of levetiracetam is on an average in the range from about 5% to about 10% per hour over a 12 hour period. In a second embodiment, after two hours, about 25 to about 60 wt. %, or about 25 to about 65 wt. %, preferably about 40 to 60 wt. %, of the total amount of the active agent is released. In a third embodiment, after four hours, about 45 to about 80 wt. %, or about 45 to about 85 wt. %, preferably about 60 to about 75 wt. % or about 55 to about 75 wt. %, of the total amount of the active agent is released. In a fourth embodiment, after six hours, about 55 to about 95 wt. %, preferably about 70 to about 90 wt. % or about 65 to about 90 wt. %, of the total amount of the active agent is released. In a fifth embodiment, after eight hours about 70 to about 100 wt. %, preferably about 80 to about 95 wt. %, of the total amount of the active agent is released. In a sixth embodiment, after twelve hours about 85 to about 100 wt. % of the total amount of the active agent is released. In a seventh embodiment, the release of levetiracetam is a biphasic release. In an eight embodiment, after one hour, about 15 wt. % to about 40 wt. %, or about 15 wt. % to about 50 wt. %, preferably about 25 wt. % to about 40 wt. % of the total amount of the active agent is released.

In a tenth aspect, there is an extended-release composition that is bioequivalent to a reference drug with a proprietary name of Keppra XR®. In a first embodiment, the patient is in a fasted state. In a second embodiment, the patient is in a non-fasted state.

In an eleventh aspect, there is a method of treatment of a seizure disorder (e.g., epilepsy), comprising administering an extended-release composition disclosed herein to an individual in need thereof, wherein the composition is administered once daily.

In various embodiments of the extended-release composition provided for herein, the levetiracetam is present in an amount of about 250 mg to about 1500 mg, preferably about 250 mg, about 500 mg, about 750 mg, about 1000 mg, or about 1500 mg.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### BRIEF DESCRIPTION OF THE DRAWINGS

FIG. 1 illustrates an example of the tablet dosage form of an extended-release levetiracetam formulation. The levetiracetam is coated with at least a hydrophobic polymer and may optionally contain a hydrophilic polymer. The reservoir particulate comprises the levetiracetam core coated with the extended-release polymer(s). The coating may be (1) complete as indicated by the levetiracetam core being within the polymeric coating. Alternately, the coating may be (2) partial as indicated by the levetiracetam core being exposed to or in contact with the extra-particulate ingredients. Also shown are agglomerates of the reservoir particulate.

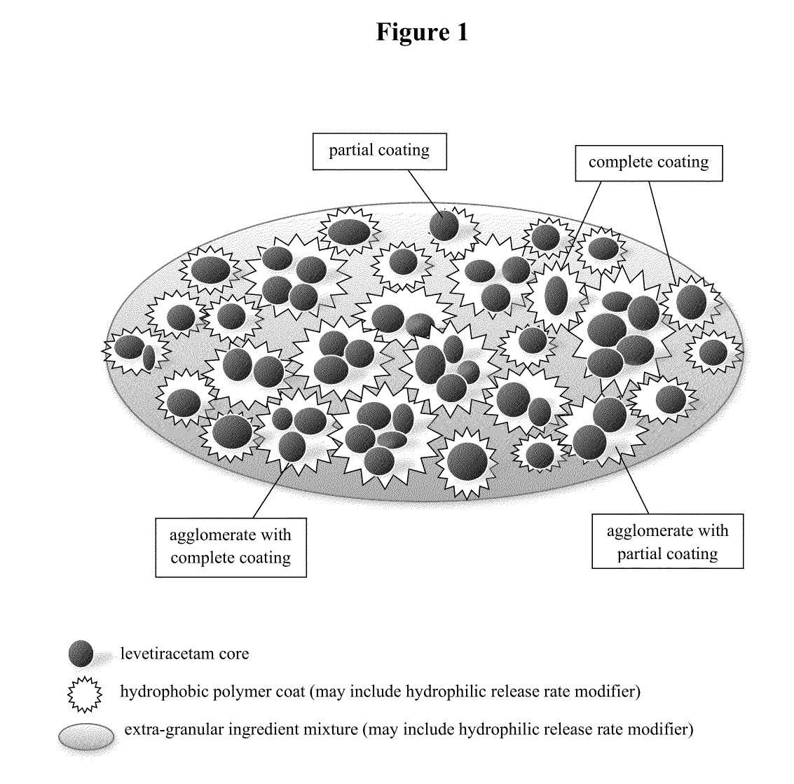

### DETAILED DESCRIPTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. Numeric ranges are inclusive of the numbers defining the range. It is to be understood that this invention is not limited to the particular methodology, protocols, and reagents described, as these may vary.

The headings provided herein are not limitations of the various aspects or embodiments of the invention which can be had by reference to the specification as a whole. Accordingly, the terms defined immediately below are more fully defined by reference to the specification as a whole.

### Definitions

The term “extended-release” herein refers to any composition which comprises levetiracetam, which is formulated to provide a gradual release of levetiracetam over a relatively longer period of time so that the concentration of levetiracetam is maintained in the blood for a longer time at a more uniform concentration than a corresponding immediate release composition comprising the same drug in the same amount. The phrase may be used interchangeably with, for example, sustained release, delayed release, controlled release, modified release, prolonged release, slow release or pulsed-release at a particular time. “Extended-release pharmaceutical compositions” means any pharmaceutical composition which is other than immediate release pharmaceutical composition.

The term “reservoir particulate” refers to one or more particles of a granulated formulation comprising an active ingredient, e.g., levetiracetam, core coated with at least one hydrophobic polymer release rate modifier.

The term “core” as used herein refers to the active ingredient, e.g., levetiracetam, without coating. The core may comprise other excipients that do not affect the active pharmaceutical ingredient (API) release rate.

The term “aqueous dispersion” refers to a water based suspension of at least one water-insoluble polymer that is substantially free of organic solvents. In some embodiments, the aqueous dispersions that find use in the present invention may further comprise a water-soluble polymer and/or other ingredients such as plasticizer, stabilizer, anti-tacking agent, etc.

The phrase “substantially free” when used with respect to the aqueous dispersion means that there are no organic solvents added to any commercially sold aqueous dispersion product. Thus, the commercial product may have de minimus quantities of organic solvents that are the result of synthesis or the manufacturing process. It will be understood by one of skill that components used in the formulations described herein may have been produced or synthesized with organic solvents and that residual amounts may be present, i.e., de minimus quantities may be present, that cannot be removed by further processing and may remain even after drying.

The phrase “release rate modifier” refers to a pharmaceutical excipient that, when present in the composition, results in an alteration to the release rate of an active ingredient, e.g., levetiracetam, as compared to the release from an identical composition in which the agent is absent, e.g., an immediate release composition.

A “dosage form” or “dosage formulation” means a unit of administration of an active agent. Examples of dosage formulations include tablets, capsules, sachets, mini-tabs, pellets, free flowing granules, beads or pills and the like. “Form” and “formulation” are to be used interchangeably and may be context dependent.

The term “matrix” refers to a cross-linked structure formed by the hydrophobic polymers in a compressed or compacted dosage form. The cross-linked structure provides a rate controlling means consisting of a hydrophobic polymer, which is provided by the reservoir particulate and, optionally, other excipients. Such embodiments will be referred to herein as matrix compositions.

The phrase “organic solvent” refers to non-aqueous liquids that find use in dissolving a hydrophobic polymer. Organic solvents include acetone, toluene, isopropyl alcohol, ethanol, methanol and the like but specifically excluding fatty alcohols and ammonia.

The phrase “controlled release layer” refers to a coating or film on the final dosage form that slows the release of active ingredient, e.g., levetiracetam. The layer may be complete or partial, i.e., some areas of the final dosage form may be uncoated.

“Bioavailability” means the extent or rate at which an active agent, e.g., levetiracetam, is absorbed into a living system or is made available at the site of physiological activity. For active agents that are intended to be absorbed into the bloodstream, bio-availability data for a given formulation may provide an estimate of the relative fraction of the administered dose that is absorbed into the systemic circulation. “Bioavailability” can be characterized by one or more pharmacokinetic parameters.

“Bioequivalence” or “bioequivalent” means the absence of a significant difference in the rate and extent to which the active agent (e.g., levetiracetam) or surrogate marker for the active agent in pharmaceutical equivalents or pharmaceutical alternatives becomes available at the site of action when administered in an appropriately designed study.

The term “hydrophobic” for purposes of the present disclosure relates to excipients, which are insoluble in water, which are water repellent, or which lack affinity toward water.

The term “biphasic release” means that there is a first period in which the active ingredient is released rapidly followed by a second period in which the active ingredient is released slowly or in a controlled manner.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ; t_{1/2} and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “t_{1/2}” refers to biological half-life: the time required for half the quantity of drug deposited in a living organism to be metabolized or eliminated by normal biological process. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC is the area under the curve of concentration versus time from time 0 to time infinity.

### Active Pharmaceutical Ingredient

The active pharmaceutical ingredient (API), e.g., levetiracetam, may be present in the reservoir particulate in an amount of between about 50% w/w to about 95% w/w of the reservoir particulate. For example, the levetiracetam may be present at any of about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% w/w, or any of about 50% to about 55%, about 55% to about 60%, about 65% to about 70%, about 70% to about 75%, about 75% to about 80%, about 80% to about 85%, about 85% to about 90%, or about 90% to about 95% w/w of the reservoir particulate. In a first embodiment, the levetiracetam is present in an amount of between about 60% w/w to about 90% w/w of the reservoir particulate. In a second embodiment, the levetiracetam is present in an amount of between about 70% w/w to about 85% w/w of the reservoir particulate. In a third embodiment, the levetiracetam is present in an amount of about 73% w/w to about 79% w/w of the reservoir particulate. In a fourth embodiment, the levetiracetam is present in an amount of about 66% w/w to about 79% w/w in the reservoir particulate. In a fifth embodiment, the levetiracetam is present in an amount of about 77% w/w of the reservoir particulate. In a sixth embodiment, the levetiracetam is present in an amount of about 78% w/w of the reservoir particulate. In a seventh embodiment, the levetiracetam is present in an amount of about 79% w/w of the reservoir particulate. In other embodiments, the levetiracetam is present in an amount of about 65% w/w to about 80% w/w, 76% w/w to about 79% w/w, or about 78% w/w to about 79% w/w of the reservoir particulate.

The levetiracetam may be present in the final dosage form in an amount of between about 30% w/w to about 95% w/w of the final dosage form. For example, the levetiracetam may be present in the final dosage form in an amount of any of about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% w/w, or any of about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, or about 90% to about 95% w/w of the final dosage form. In one embodiment, the levetiracetam is present in an amount of between about 50% w/w to about 90% w/w of the final dosage form. In another embodiment, the levetiracetam is present in an amount of between about 60% w/w to about 70% w/w, or about 63% to about 72% w/w of the final dosage form. For example, the levetiracetam may be present in an amount of any of about 60%, 61%, 62%, 63%, 64%, 65%, 66%, 67%, 68%, 69%, 70%, 71%, or 72% w/w of the final dosage form. In yet another embodiment, the levetiracetam is present in an amount of about 65% w/w of the final dosage form. In another embodiment, the levetiracetam is present in an amount of about 70% w/w of the final dosage form. In another embodiment, the levetiracetam is present in an amount of about 72% w/w of the final dosage form. In other embodiments, the levetiracetam is present in an amount of about 65% w/w to about 75% w/w, about 65% w/w to about 72% w/w, or about 70% w/w to about 72% w/w of the final dosage form.

The levetiracetam may be present in the final dosage form in an amount of between 250 mg to about 1500 mg. For example, the levetiracetam may be present in the final dosage form in an amount of any of about 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, or 1500 mg. In one embodiment, the levetiracetam may be present in the final dosage form in an amount of about 250 mg. In one embodiment, the levetiracetam may be present in the final dosage form in an amount of about 500 mg. In one embodiment, the levetiracetam may be present in the final dosage form in an amount of about 750 mg. In one embodiment, the levetiracetam may be present in the final dosage form in an amount of about 1000 mg. In one embodiment, the levetiracetam may be present in the final dosage form in an amount of about 1500 mg.

### Hydrophobic Polymers

The hydrophobic polymer used is important in controlling the release rate of levetiracetam. This hydrophobic polymer can be selected from the group consisting of cellulose ethers such as ethyl cellulose, cellulose acetate and the like, polyvinyl esters such as polyvinyl acetate, polyacrylic acid esters, butadiene styrene copolymers, methacrylic and acrylate polymers, high molecular weight polyvinyl alcohols and waxes such as fatty acids and glycerides, polymethacrylates (e.g., methacrylic acid esters neutral polymer), polyvinyl alcohol-maleic anhydride copolymers and the like, and mixtures thereof.

Ethyl cellulose—Ethyl cellulose aqueous dispersion is most preferably used. Suitable dispersions of ethyl cellulose include those available under the trade names Aquacoat® ECD-30 from FMC Corporation (Philadelphia, USA) and Surelease® from Colorcon (West Point, Pa.). Aquacoat® is an aqueous polymeric dispersion of ethyl cellulose and contains sodium lauryl sulfate and cetyl alcohol while Surelease® is an aqueous polymeric dispersion of ethyl cellulose and contains medium chain triglycerides, oleic acid, ammoniated water and fumed silica.

Ethylacrylate—methylmethacrylate copolymer aqueous dispersion is sold under the trademark Eudragit® NE 30D, Eudragit® RL and Eudragit® RL 30D.

Aminoalkyl methacrylate copolymers, for example, are marketed under the brand name of Eudragit® RS as either a dry powder or an aqueous dispersion.

An aqueous dispersion of anionic polymers with methacrylic acid as a functional group is marketed by Evonik Industries (Germany) under the brand name Eudragit® L 30 D-55.

The hydrophobic polymer is present in an amount of between about 5% w/w to about 50% w/w of the reservoir particulate. For example, the hydrophobic polymer may be present in an amount of any of about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, or 50% w/w, or any of about 5% to about 10%, about 10% to about 15%, about 15% to about 20%, about 20% to about 25%, about 25% to about 30%, about 30% to about 35%, about 35% to about 40%, about 40% to about 45%, or about 45% to about 50% w/w of the reservoir particulate. In one embodiment, the hydrophobic polymer is present in an amount of between about 5% w/w to about 40% w/w of the reservoir particulate. In another embodiment, the hydrophobic polymer is present in an amount of between about 5% w/w to about 30% w/w of the reservoir particulate. In another embodiment, the hydrophobic polymer is present in an amount of between about 5% w/w to about 20% w/w of the reservoir particulate. In another embodiment, the hydrophobic polymer is present in an amount of between about 5% w/w to about 10% w/w of the reservoir particulate. In another embodiment, the hydrophobic polymer is present in an amount of about 10% w/w to about 25% w/w of the reservoir particulate. In another embodiment, the hydrophobic polymer is present in an amount of between about 10% w/w to about 30% w/w of the reservoir particulate. In another embodiment, the hydrophobic polymer is present in an amount of between about 10% w/w to about 20% w/w of the reservoir particulate. In another embodiment, the hydrophobic polymer is present in an amount of about 13% w/w to about 19% w/w of the reservoir polymer. In another embodiment, the hydrophobic polymer is present in an amount of about 13% w/w to about 15% w/w. In yet another embodiment, the hydrophobic polymer is present in an amount of about 15% w/w of the reservoir particulate. In yet another embodiment, the hydrophobic polymer is present in amount of about 13% w/w of the reservoir particulate.

The hydrophobic polymer(s) is/are present in an amount of between about 2% w/w to about 50% w/w of the final dosage form. For example, the hydrophobic polymer(s) may be present in an amount of any of about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% w/w, or any of about 2% to about 5%, about 5% to about 10%, about 10% to about 15%, about 20% to about 25%, about 25% to about 30%, about 30% to about 35%, about 35% to about 40%, about 40% to about 45%, or about 45% to about 50% w/w of the final dosage form. In one embodiment, the hydrophobic polymer(s) is/are present in an amount of between about 5% w/w to about 30% w/w of the final dosage form. In another embodiment, the hydrophobic polymer(s) is/are present in an amount of between about 10% w/w to about 15% w/w of the final dosage form. In another embodiment, the hydrophobic polymer(s) is/are present in an amount of about 12% w/w to about 17% w/w of the final dosage form. In yet another embodiment, the hydrophobic polymer(s) is/are present in an amount of about 12% w/w of the final dosage form.

Without wishing to be bound by theory, it is believed that the hydrophobic polymers create a matrix structure that will impede the release of the active ingredient, e.g., levetiracetam. The matrix structure will remain even after all of the active ingredient has been released. Similarly, upon compression to a final dosage form, e.g., tablets, the polymers will produce an extended matrix structure that will retain the form of the final dosage form as observed in the in vitro dissolution testing.

### Hydrophilic Polymers

The term “hydrophilic polymer” as used herein is a material that is a water soluble rate controlling polymer. Suitable hydrophilic polymers include, but are not limited to water soluble polymers such as hydroxyethyl cellulose, hydroxypropyl cellulose, carboxymethyl cellulose, hydroxypropyl methylcellulose, sodium carboxymethyl cellulose, vinylpyrrolidone/vinyl acetate copolymer (for example marketed as Plasdone® S-630), polyvinyl alcohol, polyethylene glycol and the like.

Examples of hydrophilic polymers which can be used according to the present invention are: cellulose derivatives (hydroxypropyl methylcellulose, hydroxyethyl cellulose, hydroxypropyl cellulose, methylcellulose and the like); noncellulose polysaccharides (galactomannans, guar gum, carob gum, gum arabic, sterculia gum, agar, alginates and the like); polyvinylpyrrolidone; polyvinylacetate; acrylic acid polymers, such as crosslinked acrylic acid-based polymers; and a mixture of two or more of the said polymers. The hydrophilic polymers may be present in the form of a single compound or in the form of a mixture of compounds. The hydrophilic polymers preferably used according to the present invention are hydroxypropyl methylcelluloses, such as Methocel™ K or E substitution type. Examples of hydroxypropyl methylcellulose include, but are not limited to Methocel™ E3 premium LV, Methocel™ E5 premium LV, Methocel™ E6 premium LV, Methocel™ E15 premium LV, Methocel™ E50 premium LV, Methocel™ E4M premium CR, Methocel™ E10M premium CR, Methocel™ K3 premium LV, Methocel™ K100 premium LV CR, Methocel™ K4M premium CR, Methocel™ K15M premium CR, and Methocel™ K100 M premium CR.

In one aspect the hydrophilic polymer is added as a release rate modifier to the hydrophobic polymer prior to coating the levetiracetam core. The amount and specific hydrophilic polymer used may be varied to provide a desired release profile of the active ingredient, e.g., levetiracetam. Thus, if a shorter release profile is desired then either a less uniform coat is applied, a hydrophilic polymer with different release profile is used (if applied under the same conditions and manner as a previously used hydrophilic polymer) or more of the hydrophilic polymer is added to the aqueous dispersion of the hydrophobic polymer. This is well understood by one of skill in the art.

In another aspect, the hydrophilic polymer is used as a dry powder (i.e., excipient) in the extra-particulate blending. In some embodiments, the hydrophilic polymer is a hydrophilic polymer sold under the trademark Methocel™ K100M Premium CR.

The hydrophilic polymer that is added to the aqueous dispersion of the hydrophobic polymer may be the same as or different from the hydrophilic polymer used in the extra-particulate blending. For example, hydroxypropyl methylcellulose (HPMC) may be the hydrophilic polymer used in both the aqueous dispersion of the hydrophobic polymer and the extra-particulate blending. In the examples below, the hydroxypropyl methylcellulose used had differing proportions of methoxyl and hydroxpypropoxyl substitutions. Alternately, completely different hydrophilic polymers may be used, e.g., polyvinylpyrrolidone added to the hydrophobic polymer and HPMC in the extra-particulate blending or vice versa.

In one embodiment, the hydrophilic polymer is added to the aqueous dispersion of the hydrophobic polymer. The hydrophilic polymer may be a liquid, e.g., an aqueous preparation. The hydrophilic polymer may be a powder, which is then dissolved in the aqueous dispersion of the hydrophobic polymer. The hydrophilic polymer may be Pharmacoat, Methocel™ and the like. In one embodiment, the hydrophilic polymer is Hypromellose 2910, USP. In one embodiment, the hydrophilic polymer is present in an amount of about 1% w/w to about 10% w/w, more preferably about 2% w/w to about 8% w/w, even more preferably about 3% w/w to about 6% w/w and most preferably about 3% w/w to about 5% w/w in the final dosage form. In some embodiments, the hydrophilic polymer is present in an amount of about 5% w/w to about 15% w/w, or about 5% w/w to about 13% w/w in the final dosage form. In some embodiments, the hydrophilic polymer is present in an amount of about 10% w/w to about 11% w/w in the final dosage form.

In one embodiment, the hydrophilic polymer in the reservoir particulate will be present in an amount of about 0% w/w to about 19% w/w (e.g., up to about 19% w/w). For example, the hydrophilic polymer in the reservoir particulate may be present in an amount of any of about 0%, 1%; 2%, 3%, 4%, 5%, 6%, 7%, 8%, 9%, 10%, 11%, 12%, 13%, 14%, 15%, 16%, 17%, 18%, or 19% w/w, or any of 0% to about 5%, about 5% to about 10%, about 10% to about 15%, or about 15% to about 19% w/w. In one embodiment, the hydrophilic polymer is present in an amount of about 0% w/w to about 15% w/w. In one embodiment, the hydrophilic polymer is present in an amount of about 0% w/w to about 10% w/w. In one embodiment, the hydrophilic polymer is present in an amount of about 0% w/w to about 5% w/w. In one embodiment, the hydrophilic polymer is present in an amount of about 5% w/w to about 19% w/w. In one embodiment, the hydrophilic polymer is present in an amount of about 5% w/w to about 15% w/w. In one embodiment, the hydrophilic polymer is present in an amount of about 5% w/w to about 10% w/w. In one embodiment, the hydrophilic polymer is present in an amount of about 3% w/w to about 13% w/w. In one embodiment, the hydrophilic polymer is present in an amount of about 3% w/w to about 5% w/w. In one embodiment, the hydrophilic polymer is present in an amount of about 4% w/w. In one embodiment, the hydrophilic polymer is present in an amount of about 3% w/w. In one embodiment, the hydrophilic polymer is Hypromellose 2910, USP.

In one embodiment, the hydrophilic polymer is a powder, which is used during extra-particulate blending. In one embodiment, the hydrophilic polymer is Hypromellose 2208, USP. In one embodiment, the hydrophilic polymer is present in an amount of about 0% w/w to about 19% w/w (e.g., up to about 19% w/w), more preferably about 3% w/w to about 9% w/w, even more preferably about 4% w/w to about 8% w/w and most preferably about 5% w/w to about 7% w/w in the final dosage form. For example, the hydrophilic polymer used in extra-particulate blending may be present in an amount of any of about 0%, 1%, 2%, 3%, 4%, 5%, 6%, 7%, 8%, 9%, 10%, 11%, 12%, 13%, 14%, 15%, 16%, 17%, 18%, or 19% w/w, or any of 0% to about 5%, about 5% to about 10%, about 6% w/w to about 8% w/w, about 10% to about 15%, or about 15% to about 19% w/w in the final dosage form. In one embodiment, the hydrophilic polymer used during extra-particulate blending is present in an amount of about 6% w/w in the final dosage form. In one embodiment, the hydrophilic polymer used during extra-particulate blending is present in an amount of about 8% w/w in the final dosage form.

The hydrophilic polymer(s) is/are present (when used in both the reservoir particulate and the extra-particulate blending) in a total amount of between about 0% w/w to about 38% w/w of the final dosage form. For example, the hydrophilic polymer(s) may be present when used in both the reservoir particulate and the extra-particulate blending in a total amount of any of about 0%, 1%; 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, or 38% w/w, or any of 0% to about 5%, about 5% to about 10%, about 10% to about 15%, about 15% to about 20%, about 20% to about 25%, about 25% to about 30%, about 30% to about 35%, or about 35% to about 38% w/w of the final dosage form. In one embodiment, the hydrophilic polymer(s) is/are present in an amount of between about 3% w/w to about 30% w/w of the final dosage form. In another embodiment, the hydrophilic polymer(s) is/are present in an amount of between about 5% w/w to about 15% w/w of the final dosage form. In yet another embodiment, the hydrophilic polymer(s) is/are present in an amount of about 3% to about 5% w/w of the final dosage form. In yet another embodiment, the hydrophilic polymer(s) is/are present in a total amount of about 5-15% w/w of the final dosage form. In another embodiment, the hydrophilic polymer(s) is/are present in a total amount of 5% w/w to about 13% w/w of the final dosage form. In another embodiment, the hydrophilic polymer(s) is/are present in a total amount of about 10% w/w to about 11% w/w of the final dosage form. In one embodiment, the hydrophilic polymer(s) is/are present in a total amount of about 10% of the final dosage form. In another embodiment, the hydrophilic polymer(s) is/are present in a total amount of about 11% w/w of the final dosage form.

### Aqueous Dispersions of Hydrophobic Polymers

The aqueous dispersions that find use in the present invention can be prepared using well known methods in the art. See, for example, U.S. Pat. Nos. 4,123,403 and 4,502,888; see also Iqbal et al., J. Chem. Soc. Pak. 33(5):634-639 (2011). When the aqueous dispersion is produced de novo, i.e., not a commercial product, it is produced without organic solvents. Thus, the aqueous dispersion is free of organic solvents. Alternatively, a commercial aqueous dispersion of a hydrophobic polymer may be used. Specifically, Eudragit® L-30D, Eudragit® NE30D, Aquacoat® ECD-30, Surelease® E-7, Eudragit® RS 30D, and/or Eudragit® RL 30D may be used. Specifically contemplated is the use of a commercially available aqueous dispersion that is substantially free of organic solvents. In another embodiment, the aqueous dispersion is free of organic solvents.

The aqueous dispersion of hydrophobic polymer may be used for controlling release rate of levetiracetam from the reservoir particulate and/or dosage form.

The water insoluble hydrophobic polymer is present in an amount from about 2% w/w to about 50% w/w in total weight of the final composition, and optionally containing a release rate modifier such as a hydrophilic excipient or a pore former in an amount from about 0% w/w to about 19% w/w in total weight of the final composition. For example, the water insoluble hydrophobic polymer may be present in an amount of any of about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%, or any of about 2% to about 5%, about 5% to about 10%, about 10% to about 15%, about 15% to about 20%, about 20% to about 25%, about 25% to about 30%, about 30% to about 35%, about 35% to about 40%, about 40% to about 45%, or about 45% to about 50% w/w in total weight of the final composition. For example, optionally a release rate modifier such as a hydrophilic excipient or a pore former may be present in an amount of any of about 0%, 1%, 2%, 3%, 4%, 5%, 6%, 7%, 8%, 9%, 10%, 11%, 12%, 13%, 14%, 15%, 16%, 17%, 18%, or 19%, or any of 0% to about 5%, about 5% to about 10%, about 10% to about 15%, or about 15% to about 19% w/w in total weight of the final composition. The release rate modifier in the rate controlling hydrophobic polymer may be selected from copolyvidone, polyvinylpyrrolidone (PVP), polyethylene glycols, hydroxyethyl cellulose, hydroxypropyl methylcellulose, hydroxypropyl cellulose, etc. In particular embodiments, the aqueous dispersion contains ethyl cellulose.

### Excipients

The conventional excipients according to the present invention are those excipients which are commonly used in the art and known to any person skilled in the art. These include, but are not limited to, fillers, binders, lubricants, plasticizers, glidants and the like.

Examples of fillers or diluents include, but are not limited to, corn starch, lactose, sucrose, microcrystalline cellulose, kaolin, mannitol, dextrose, lactose, sorbitol, dicalcium phosphate, calcium carbonate, sodium chloride, maltitol, xylitol and the like. In some embodiments, a filler, for example, lactose, may be present in an amount up to about 12% w/w (0% to about 12% w/w), for example, about 5% w/w to about 12% w/w. In some embodiments, lactose is present in an amount of any of about 0%, 1%, 2%, 3%, 4%, 5%, 6%, 7%, 8%, 9%, 10%, 11%, or 12% w/w) of the formulation, e.g., the final dosage form.

Examples of binders include, but are not limited to methylcellulose, hydroxypropyl cellulose, hydroxyethyl cellulose, hydroxypropyl methylcellulose, sodium carboxymethylcellulose, polyvinylpyrrolidone, sucrose, starch, ethylcellulose, acacia, gelatin, gum arabic, copovidone, polyvinyl alcohol, pullulan, agar, tragacanth, sodium alginate, alginic acid, and the like; glycerides such as for example mono-, di- or triglycerides such as, e.g., stearin, palmitin, laurin, myristin, hydrogenated castor or cottonseed oils, glyceryl palmitostearate, glyceryl behenate and the like; fatty acids and alcohols such as, e.g., stearic, palmitic or lauric acids, stearyl, cetyl or cetosteryl alcohols and the like; and waxes such as for example white wax, bees wax, carnauba wax and the like.

Examples of lubricants and glidants include, but are not limited, to stearates and stearic acid, silicone fluid, talc, waxes, oils, colloidal silicon dioxide, sodium stearyl fumarate, polyethylene glycols, hydrogenated vegetable oil, glyceryl behenate, magnesium trisilicate, microcrystalline wax, yellow beeswax, white beeswax and the like. In some embodiments the stearate is magnesium stearate. In some embodiments, lubricant(s) and/or glidant(s) may be present in an amount of about 1% w/w to about 4% w/w (e.g., any of about 1%, 2%, 3% or 4% w/w) of the formulation, e.g., the final dosage form. In some embodiments, colloidal silicon dioxide may be present in an amount of about 1% w/w to about 4% w/w (e.g., about 1%, 2%, 3%, or 4% w/w) of the formulation, e.g., the final dosage form. In some embodiments, a stearate, for example, magnesium stearate, may be present in an amount of about 1% w/w to about 4% w/w (e.g., about 1%, 2%, 3%, or 4% w/w) of the formulation, e.g., the final dosage form. In some embodiments, a combination of colloidal silicon dioxide and magnesium stearate is included at a total combined amount of about 1% w/w to about 4% w/w (e.g., about 1%, 2%, 3%, or 4% w/w) of the formulation, e.g., the final dosage form. In one amount, the formulation includes about 1% w/w colloidal silicon dioxide and about 3% w/w magnesium stearate. In another embodiment, the formulation includes about 2% w/w magnesium stearate and no colloidal silicon dioxide. In another embodiment, the formulation includes about 3% w/w magnesium stearate and no colloidal silicon dioxide. In some embodiments, the composition does not include lactose. In some embodiments, the composition includes colloidal silicon dioxide and/or magnesium stearate and does not include lactose.

### Methodology

The present invention further relates to processes for manufacturing pharmaceutical formulations of the present invention, wherein an aspect is a process comprising:
1) Sifting levetiracetam and, optionally, other excipients such as diluents, etc. through a sieve and mixing;
2) Granulating step 1) materials using an aqueous dispersion of a hydrophobic polymer;
3) Removing water, e.g., drying, to form a reservoir particulate;
4) Sifting the dried reservoir particulate through a sieve to remove large agglomerates. Then, sifting extraparticulate excipients through a sieve;
5) Blending sifted reservoir particulate and extra-particulate excipients and adding a lubricant to the blend;
6) Producing dosage form, e.g., compressing the final lubricated blend into tablets or filling into capsules or sachets; and
7) Optionally, coating tablets or capsules with hydrophobic, lipophilic, or hydrophilic materials along with other coating adjuvants.

The granulation step of the above method may provide for the removal of water, e.g., drying, simultaneously with the coating of the levetiracetam to form the reservoir particulate. In such cases, steps 2 and 3 merge into a single step.

The levetiracetam core is levetiracetam or a pharmaceutically acceptable salt, solvate, hydrate, crystalline form or non-crystalline form thereof, or any combination thereof.

In another aspect, disclosed herein is a controlled release pharmaceutical composition for oral administration prepared by a process which comprises:
a) preparing a reservoir particulate comprising a levetiracetam core and a coating from an aqueous dispersion of a hydrophobic polymer.

In one embodiment, the method comprises:
a) preparing a reservoir particulate comprising a levetiracetam core and a coating from an aqueous dispersion of a hydrophobic polymer; and
b) blending the reservoir particulate of step (a) with a release rate-modifier comprising hydrophilic polymer(s) and one or more pharmaceutically acceptable excipients.

In an embodiment, the levetiracetam core is levetiracetam or a pharmaceutically acceptable salt, solvate, hydrate, crystalline form or non-crystalline form thereof.

### Reservoir Particulate Formation

The reservoir particulate may be formed using any method known by one of skill in the art. For example, the reservoir particulate can be prepared by wet granulation, melt granulation, spheronization, extrusion, hot fusion and the like, including combinations thereof. In a preferred embodiment, the reservoir particulate comprising levetiracetam is prepared by wet granulation. Specifically, contemplated is the use of a fluid bed granulator.

Levetiracetam crystals are combined with an aqueous dispersion of a hydrophobic polymer in a fluid bed granulator. The levetiracetam is agitated and the hydrophobic polymer is layered onto the crystals. The reservoir particulates are obtained as the hydrophobic polymer is layered onto the crystals by drying off the water. Agglomerates may be formed.

While in the process it is desirable to layer or coat the entire surface of the crystal it is recognized that some of the crystal surface may not be covered. In other words, the coating may be partial.

The reservoir particulate is then dried by any suitable means known to one of skill in the art. Such means include, but are not limited to, spray drying, vacuum drying, oven drying or fluid bed drying. The reservoir particulate is dried to a moisture content of about 0.05-5% w/w as measured by weight loss using the loss on drying (LOD) method at 105° C. (U.S. Pharmacopeia <731> Loss on Drying). Preferably the reservoir particles are dried to less than 1.0% w/w water content.

In an embodiment, the levetiracetam core consists of levetiracetam. In another embodiment, the levetiracetam core comprises levetiracetam. In yet another embodiment, the levetiracetam core consists essentially of levetiracetam.

In an embodiment, the reservoir particulate comprises a levetiracetam core and a hydrophobic polymer coating. In another embodiment, the reservoir particulate comprises a levetiracetam core comprising levetiracetam and a hydrophobic polymer coating. In another embodiment, the reservoir particulate comprises a levetiracetam core comprising levetiracetam and a coating comprising a hydrophilic polymer and a hydrophobic polymer. In yet another embodiment, the reservoir particulate comprises a levetiracetam core comprising levetiracetam and a coating consisting essentially of a hydrophilic polymer and a hydrophobic polymer. In a further embodiment, the reservoir particulate comprises a levetiracetam core comprising levetiracetam and a coating consisting of a hydrophilic polymer and a hydrophobic polymer. In an embodiment, the reservoir particulate comprises a levetiracetam core consisting of levetiracetam and a coating consisting of a hydrophilic polymer and a hydrophobic polymer.

In the various aspects, the hydrophobic polymer is coated onto the levetiracetam core as an aqueous dispersion. In some embodiments, the aqueous dispersion consists of the hydrophobic polymer. In an embodiment, the aqueous dispersion of the hydrophobic polymer further contains a hydrophilic polymer.

In various embodiments, the aqueous dispersion of the hydrophobic polymer is an aqueous dispersion of ethyl cellulose. In some embodiments, the aqueous dispersion of the hydrophobic polymer is Surelease® E-7 19040 (Colorcon).

### Extra-Particulate Blending

The dried reservoir particulates may be blended with additional ingredients such as pharmaceutically acceptable excipients. The ingredients may be selected from, but are not limited to, release rate modifiers, fillers (i.e., diluents), binders, lubricants, plasticizers, surfactants, glidants and the like. In some embodiments, the ingredients include, but are not limited to, colloidal silicon dioxide, magnesium stearate, sodium lauryl sulfate, and the like.

In certain embodiments, the pharmaceutically acceptable excipients are a hydrophilic polymer, a lubricant and a diluent. In an embodiment, the pharmaceutically acceptable excipients are lactose, magnesium stearate and hydroxypropyl methylcellulose. In a specific embodiment, the pharmaceutically acceptable excipients are lactose, magnesium stearate and a hydrophilic polymer sold under the trademark Methocel™ K100M Premium CR.

In some embodiments, the pharmaceutically acceptable excipients include, but are not limited to, colloidal silicon dioxide, magnesium stearate, sodium lauryl sulfate, and the like. In some embodiments, the pharmaceutically acceptable excipients consist essentially of colloidal silicon dioxide, magnesium stearate, lactose, and a hydrophilic polymer.

In some embodiments, the release rate modifier is a hydrophilic polymer. In embodiments, the hydrophilic polymer is a hydrophilic polymer sold under the trademark Methocel™ K100M Premium CR.

### Dosage Form

Once the reservoir particulates and the extra-particulate pharmaceutically acceptable excipients are blended the resultant blend is (1) compressed into tablets, mini-tabs, pellets, beads or pills, (2) filled into capsules or sachets, or (3) provided as free flowing granules.

Equipment suitable for processing the pharmaceutical formulation of the present invention includes any one or a combination of mechanical sifters, blenders, roller compactors, granulators (high shear mixer, low shear mixer or fluid bed granulator), fluid bed dryers, compression machines, rotating bowls or coating pans, etc. The fluid bed granulators may be either top, side (i.e., tangential) or bottom spray configured.

In some embodiments, a formulation (e.g., dosage form) prepared as described herein may include about 66% w/w to about 79% w/w levetiracetam and about 13% w/w to about 19% w/w hydrophobic polymer (e.g., ethyl cellulose) in the reservoir particulate. In one embodiment, a formulation (e.g., dosage form) prepared as described herein may include about 76% w/w to about 79% w/w levetiracetam and about 13% w/w to about 14% w/w hydrophobic polymer (e.g., ethyl cellulose) in the reservoir particulate. In one embodiment, a formulation (e.g., dosage form) prepared as described herein may include about 76% w/w levetiracetam and about 14% w/w hydrophobic polymer (e.g., ethyl cellulose) in the reservoir particulate. In one embodiment, a formulation (e.g., dosage form) prepared as described herein may include about 78% w/w levetiracetam and about 13% w/w hydrophobic polymer (e.g., ethyl cellulose) in the reservoir particulate. In one embodiment, a formulation (e.g., dosage form) prepared as described herein may include about 79% w/w levetiracetam and about 13% w/w hydrophobic polymer (e.g., ethyl cellulose) in the reservoir particulate.

In some embodiments, a formulation (e.g., dosage form) prepared as described herein may include about 66% w/w to about 79% w/w levetiracetam, about 13% w/w to about 19% w/w hydrophobic polymer (e.g., ethyl cellulose), and about 3% w/w to about 13% w/w hydrophilic polymer (e.g., hydroxypropyl methylcellulose) in the reservoir particulate. In one embodiment, a formulation (e.g., dosage form) prepared as described herein may include about 76% w/w to about 79% w/w levetiracetam, about 13% w/w to about 14% w/w hydrophobic polymer (e.g., ethyl cellulose), and about 3% w/w to about 5% w/w hydrophilic polymer (e.g., hydroxypropyl methylcellulose) in the reservoir particulate. In one embodiment, a formulation (e.g., dosage form) prepared as described herein may include about 76% w/w levetiracetam, about 14% w/w hydrophobic polymer (e.g., ethyl cellulose), and about 5% w/w hydrophilic polymer (e.g., hydroxypropyl methylcellulose) in the reservoir particulate. In one embodiment, a formulation (e.g., dosage form) prepared as described herein may include about 78% w/w levetiracetam, about 13% w/w hydrophobic polymer (e.g., ethyl cellulose), and about 4% hydrophilic polymer (e.g., hydroxypropyl methylcellulose) in the reservoir particulate. In one embodiment, a formulation (e.g., dosage form) prepared as described herein may include about 79% w/w levetiracetam and about 13% w/w hydrophobic polymer (e.g., ethyl cellulose), and about 3% w/w hydrophilic polymer (e.g., hydroxypropyl methylcellulose) in the reservoir particulate.

In some embodiments, a formulation (e.g., dosage form) prepared as described herein may include about 63% w/w to about 72% w/w levetiracetam, about 12% w/w to about 17% w/w hydrophobic polymer (e.g., ethyl cellulose), 0% to about 13% w/w hydrophilic polymer (e.g., hydroxypropyl methylcellulose), 0% to about 12% w/w filler (e.g., lactose), and about 1% w/w to about 3% w/w glidant and/or lubricant (e.g., colloidal silicon dioxide; magnesium stearate). In some embodiments, a formulation (e.g., dosage form) prepared as described herein may include about 70% w/w to about 72% w/w levetiracetam, about 12% w/w hydrophobic polymer (e.g., ethyl cellulose), about 10% w/w to about 11% w/w hydrophilic polymer (e.g., hydroxypropyl methylcellulose), and about 2% w/w to about 3% w/w glidant and/or lubricant (e.g., magnesium stearate). In one embodiment, a formulation (e.g., dosage form) prepared as described herein may include about 65% w/w levetiracetam, about 12% w/w hydrophobic polymer (e.g., ethyl cellulose), about 10% w/w hydrophilic polymer (e.g., hydroxypropyl methylcellulose), about 5% w/w filler (e.g., lactose), and about 4% w/w glidant and/or lubricant (e.g., about 1% w/w colloidal silicon dioxide, about 3% magnesium stearate). In one embodiment, a formulation (e.g., dosage form) prepared as described herein may include about 72% w/w levetiracetam, about 12% w/w hydrophobic polymer (e.g., ethyl cellulose), about 10% w/w hydrophilic polymer (e.g., hydroxypropyl methylcellulose), and about 2% w/w glidant and/or lubricant (e.g., about 2% w/w magnesium stearate). In one embodiment, a formulation (e.g., dosage form) prepared as described herein may include about 70% w/w levetiracetam, about 12% w/w hydrophobic polymer (e.g., ethyl cellulose), about 11% w/w hydrophilic polymer (e.g., hydroxypropyl methylcellulose), and about 3% w/w glidant and/or lubricant (e.g., about 3% w/w magnesium stearate).

### Bioequivalency

In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “Guidance For Industry Bioavailability And Bioequivalence Studies For Orally Administered Drug Products-General Considerations” available from the U.S. Department of Health and Human Services (DHHS), Food and Drug Administration (FDA), Center for Drug Evaluation and Research (CDER) March 2003 Revision 1; and “Guidance For Industry Statistical Approaches To Establishing Bioequivalence” DHHS, FDA, CDER, January 2001, both of which are incorporated herein in their entirety.

In an embodiment, bioequivalence of a composition to a reference drug is determined by an in vivo pharmacokinetic study to determine a pharmacokinetic parameter for the active agent composition. Specifically, bioequivalence can be determined by an in vivo pharmacokinetic study comparing a pharmacokinetic parameter for the two compositions. A pharmacokinetic parameter for the active agent composition or the reference drug can be measured in a single or multiple dose bioequivalence study using a replicate or a nonreplicate design. For example, the pharmacokinetic parameters for active agent composition of the present invention and for a reference drug can be measured in a single dose pharmacokinetic study using a two-period, two-sequence crossover design. Alternately, a four-period, replicate design crossover study may also be used. Single doses of the test composition and reference drug are administered and blood or plasma levels of the active agent are measured over time. Pharmacokinetic parameters characterizing rate and extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

Under U.S. FDA guidelines, two products (e.g., an inventive levetiracetam formulation and Keppra XR® 500 mg) or methods (e.g., dosing under non-fasted versus fasted conditions) are bioequivalent if the 90% Confidence Interval (CI) limits for a ratio of the geometric mean of logarithmic transformed AUC_{0-∞}, AUC₀₋ₜ, and Cₘₐₓ for the two products or two methods are about 0.80 to about 1.25.

In another embodiment, bioequivalence is determined according to the European Medicines Agency (EMEA) document “Note for Guidance on the Investigation of Bioavailability and Bioequivalence”, issued Jul. 26, 2001, available from EMEA.

To show bioequivalency between two compositions or administration conditions pursuant to Europe's EMEA guidelines, the 90% CI limits for a ratio of the geometric mean of logarithmic transformed AUC_{0-∞} and AUC₀₋ₜ for the two products or methods are about 0.80 to about 1.25. The 90% CI limits for a ratio of the geometric mean of logarithmic transformed Cₘₐₓ for the two products or methods can have a wider acceptance range when justified by safety and efficacy considerations. For example the acceptance range can be about 0.70 to about 1.43, specifically about 0.75 to about 1.33, and more specifically about 0.80 to about 1.25.

In one embodiment, in a given experiment, an active agent composition is considered to be bioequivalent to the reference drug if the Test/Reference ratio for the geometric mean of logarithmic transformed AUC_{0-∞}, AUC₀₋ₜ, and Cₘₐₓ ratio along with its corresponding lower and upper 90% CI limits are all within a lower limit of about 0.80 and an upper limit of about 1.25. Thus, for direct comparison between an inventive active agent composition and a reference drug, the pharmacokinetic parameters for the active agent composition and the reference drug can be determined side-by-side in the same pharmacokinetic study.

In some embodiments a single dose bioequivalence study is performed under non-fasted or fasted conditions.

In other embodiments, the single dose bioequivalence study is conducted between the active agent composition and the reference drug using the strength specified by the FDA in Approved Drug Products With Therapeutic Equivalence Evaluations (Orange Book).

In some embodiments, an in vivo bioequivalence study is performed to compare all active agent compositions with corresponding strengths of the reference drug (e.g., 500 or 750 mg of the active agent). In other embodiments, an in vivo bioequivalence study is performed only for the active agent composition of the present invention at the strength of the reference listed drug product (e.g., the highest approved strength) and at the other lower or higher strengths, the inventive compositions meet an appropriate in vitro dissolution test.

### Methods of Treatment

Methods of treatment with the extended-release compositions disclosed herein are provided. In some embodiments, a method of treatment is provided that includes administration of an extended-release composition (e.g., a dosage form that includes a reservoir particulate as described herein, with a core that contains levetiracetam, a pharmaceutically acceptable salt, solvate, hydrate, crystalline form, non-crystalline form or combination thereof, coated with an aqueous dispersion that contains at least one hydrophobic polymer, wherein the aqueous dispersion is free or substantially free of organic solvents) to an individual in need thereof. In some embodiments, the extended-release composition is administered for the treatment of a seizure disorder (e.g., epilepsy). In some embodiments, the extended-release composition is administered once daily.

In some embodiments, the extended-release pharmaceutical composition contains levetiracetam (e.g., levetiracetam, a pharmaceutically acceptable salt, solvate, hydrate, crystalline form, non-crystalline form or combination thereof) at a concentration of about 30% w/w to about 95% w/w. In some embodiments, the levetiracetam is present at a concentration of about 50% w/w to about 90% w/w.

In some embodiments, the extended-release pharmaceutical composition contains a hydrophobic polymer at a concentration of about 2% w/w to 50% w/w. In some embodiments, the hydrophobic polymer is present at a concentration of about 5% w/w to about 30% w/w.

In some embodiments, the extended-release pharmaceutical composition contains at least one hydrophilic polymer at a concentration of about 5% w/w to about 19% w/w. In some embodiments, the hydrophilic polymer is present at a concentration of about 5% w/w to about 13% w/w.

In some embodiments, the extended-release pharmaceutical composition contains levetiracetam (e.g., levetiracetam, a pharmaceutically acceptable salt, solvate, hydrate, crystalline form, non-crystalline form or combination thereof) at a concentration of about 30% w/w to about 95% w/w, and the hydrophobic polymer(s) at a concentration of about 2% w/w to about 50% w/w. In some embodiments, the extended-release pharmaceutical composition contains levetiracetam is at a concentration of about 30% w/w to about 95% w/w, the hydrophobic polymer(s) at about 2% w/w to about 50% w/w, and at least one hydrophilic polymer is present at a concentration up to about 19% w/w. In some embodiments, the levetiracetam is present at a concentration of about 50% w/w to about 90% w/w, the hydrophobic polymer(s) is(are) present at about 5% w/w to about 30% w/w, and the hydrophilic polymer(s) is(are) present at a concentration of about 5% w/w to about 19% w/w.

In some embodiments, the extended-release pharmaceutical composition releases about 85 wt. % to about 100 wt. % of the levetiracetam (e.g., levetiracetam, a pharmaceutically acceptable salt, solvate, hydrate, crystalline form, non-crystalline form or combination thereof) contained therein over a period of about 12 hours after introduction of the dosage form into the dissolution medium when tested in 900 mL of pH 6.0 phosphate buffer maintained at 37° C. using a basket method (USP Apparatus 1) at 100 rpm.

In some embodiments, the extended-release pharmaceutical composition is bioequivalent to a reference drug with a proprietary name of Keppra XR® when administered to a patient in a fasted or non-fasted state.

In some embodiments, about 250 mg to about 1500 mg of levetiracetam (e.g., levetiracetam, a pharmaceutically acceptable salt, solvate, hydrate, crystalline form, non-crystalline form or combination thereof) is administered per day (e.g., once daily) in a method of treatment as described herein. In one embodiment, about 500 mg is administered. In another embodiment, about 750 mg is administered.

In one embodiment, the extended-release pharmaceutical composition contains levetiractem at a concentration of about 72% w/w, at least one hydrophobic polymer at a concentration of about 12% w/w, and at least one hydrophilic polymer at a concentration of about 10% w/w, formulated in a tablet that comprises about 750 mg of levetiracetam.

In one embodiment, the extended-release pharmaceutical composition contains levetiractem at a concentration of about 70% w/w, at least one hydrophobic polymer at a concentration of about 12% w/w, and at least one hydrophilic polymer at a concentration of about 11% w/w, formulated in a tablet that comprises about 500 mg of levetiracetam.

In any of the embodiments of the methods of treatment described herein, the hydrophobic polymer may be ethyl cellulose and the hydrophilic polymer may be hydroxypropyl methylcellulose.

In the experimental disclosure which follows, the following abbreviations apply: eq (equivalents); M (Molar); μM (micromolar); N (Normal); mol (moles); mmol (millimoles); pmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); hr (hours); min (minutes); sec (seconds); msec (milliseconds).

### EXAMPLES

The present invention is described in further detail in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

### Example 1

### Extended-Release Composition

This example illustrates an embodiment of an extended-release composition that is free of hydrophilic polymers.

TABLE 1
Composition

| S. No. | Ingredient | Quantity (% w/w) |
|---|---|---|
| 1 | Levetiracetam (Waterstone) | 63 |
| 2 | Ethyl cellulose (from aqueous dispersion, | 16 |
| | quantity of solid content) | |
| 3 | Other solids in ethyl cellulose aqueous | 5 |
| | dispersion | |
| 4 | Water (used to dilute ethyl cellulose | q.s. |
| | dispersion prior to coating) | |
| 5 | Lactose | 12 |
| 6 | Colloidal Silicon Dioxide | 1 |
| 7 | Magnesium Stearate | 3 |
| | Total | 100 |

q.s. means a sufficient amount

Levetiracetam was granulated with aqueous ethyl cellulose dispersion with or without other ingredients such as plasticizers and subsequently dried in a fluid-bed unit to produce a reservoir particulate that is free of hydrophilic polymers. The ethyl cellulose used was Surelease® E-7 19040 (Colorcon) and was diluted with water to a final solids content of about 10-15% w/w. The reservoir particulate may be filled into capsules or sachets.

The reservoir particulate was then blended with lactose and colloidal silicon dioxide and lubricated with magnesium stearate. Colloidal silicon dioxide may be blended before, after, or at the same time as the lactose. The final blend was then filled into capsules or sachets, or compressed into tablets. For example, the blend may be formulated in a dosage form at a strength of 750 mg levetiracetam.

### Example 2

### Extended-Release Composition with Hydrophilic Polymer

The following example illustrates an embodiment of an extended-release composition that incorporates hydrophilic polymers in the reservoir particulate.

TABLE 2
Composition

| S. No. | Ingredient | Quantity (% w/w) |
|---|---|---|
| 1 | Levetiracetam | 65 |
| 2 | Ethyl cellulose (from aqueous dispersion, | 15 |
| | quantity in solid content) | |
| 3 | Other solids in ethyl cellulose aqueous | 5 |
| | dispersion | |
| 4 | Hydroxypropyl methylcellulose | 5 |
| 5 | Water | q.s. |
| 6 | Lactose | 7 |
| 7 | Magnesium Stearate | 3 |
| | Total | 100 |

The reservoir particulate is made as in Example 1 except that a release rate modifier, with or without plasticizers, is added to the aqueous ethyl cellulose dispersion. The release rate modifier may be a hydrophilic excipient, preferably a hydrophilic polymer, for example, hydroxypropyl methylcellulose. In this example the release rate modifier is a hydrophilic polymer sold under the trademark Methocel™ E5 Premium LV.

The aqueous ethyl cellulose dispersion with the release rate modifier is used to coat the Levetiracetam to produce a reservoir particulate as in Example 1. The reservoir particulate may be filled into capsules or sachets.

The dried reservoir particulate is blended with lactose and lubricated with magnesium stearate. The final blend is then filled into capsules or sachets, or compressed into tablets. For example, the blend may be formulated in a dosage form at a strength of 750 mg levetiracetam.

### Example 3

### Extra-Particulate Release Rate Modifier

This example illustrates an embodiment of an extended-release composition that incorporates hydrophilic polymers in the reservoir particulate and an extra-particulate release rate modifier.

TABLE 3
Composition

| S. No. | Ingredient | Quantity (% w/w) |
|---|---|---|
| 1 | Levetiracetam | 64 |
| 2 | Ethyl cellulose (from aqueous dispersion, | 14 |
| | quantity in solid content) | |
| 3 | Other solids in ethyl cellulose aqueous | 5 |
| | dispersion | |
| 4 | Hydroxypropyl methylcellulose | 3 |
| | (in reservoir) | |
| 5 | Water | q.s. |
| 6 | Lactose | 4 |
| 7 | Hydroxypropyl methylcellulose (extra- | 6 |
| | granular) | |
| 8 | Colloidal Silicon Dioxide | 1 |
| 9 | Magnesium Stearate | 3 |
| | Total | 100 |

The reservoir particulate was produced as in Example 2. The reservoir particulate may be filled into capsules or sachets.

The reservoir particulate was subsequently blended with lactose and colloidal silicon dioxide and a release rate modifier then lubricated with magnesium stearate. Lactose, the release rate modifier, and colloidal silicon dioxide may be blended in any order, separately or together, with magnesium stearate added last. The release rate modifier blended with the reservoir particulate was a hydrophilic polymer, hydroxypropyl methylcellulose, sold under the trademark Methocel™ K100M Premium CR. The final blend was then filled into capsules or sachets, or compressed into tablets. For example, the blend may be formulated in a dosage form at a strength of 750 mg levetiracetam.

### Example 4

### Extra-Particulate Release Rate Modifier

This example illustrates an embodiment similar to that of Example 3 but with different pharmaceutically acceptable excipients (different grade of lactose).

TABLE 4
Composition

| S. No. | Ingredient | Quantity (% w/w) |
|---|---|---|
| 1 | Levetiracetam | 65 |
| 2 | Ethyl cellulose (from aqueous dispersion, | 12 |
| | quantity in solid content) | |
| 3 | Other solids in ethyl cellulose aqueous | 4 |
| | dispersion | |
| 4 | Hydroxypropyl methylcellulose | 4 |
| | (in reservoir) | |
| 5 | Water | q.s. |
| 6 | Lactose | 5 |
| 7 | Hydroxypropyl methylcellulose | 6 |
| | (extra-granular) | |
| 8 | Colloidal Silicon Dioxide | 1 |
| 9 | Magnesium Stearate | 3 |
| | Total | 100 |

The reservoir particulate was produced as in Example 2. The reservoir particulate may be filled into capsules or sachets.

The reservoir particulate was subsequently blended with lactose, colloidal silicon dioxide and a release rate modifier, hydroxypropyl methylcellulose, then lubricated with magnesium stearate. The release rate modifier was a hydrophilic polymer sold under the trademark Methocel™ K100M Premium CR. The final blend was then filled into capsules or sachets, or compressed into tablets. For example, the blend may be formulated in a dosage form at a strength of 750 mg levetiracetam.

### Example 5

### Extended-Release Composition with Hydrophilic Polymer

The following example illustrates an embodiment of an extended-release composition that incorporates hydrophilic polymers in the reservoir particulate.

TABLE 5
Composition

| S. No. | Ingredient | Quantity (% w/w) |
|---|---|---|
| 1 | Levetiracetam | 70 |
| 2 | Ethyl cellulose (from aqueous dispersion, | 17 |
| | quantity in solid content) | |
| 3 | Other solids in ethyl cellulose aqueous | 6 |
| | dispersion | |
| 4 | Hydroxypropyl methylcellulose | 5 |
| 5 | Water | q.s. |
| 6 | Magnesium Stearate | 2 |
| | Total | 100 |

The reservoir particulate was made as in Example 1 except that a release rate modifier was added to the aqueous ethyl cellulose dispersion. In this example the release rate modifier was a hydrophilic polymer sold under the trademark Methocel™ E5 Premium LV.

The aqueous ethyl cellulose dispersion with the release rate modifier was used to coat the levetiracetam to produce a reservoir particulate as in Example 1. The reservoir particulate may be filled into capsules or sachets.

The dried reservoir particulate was lubricated with magnesium stearate. The final blend was then filled into capsules or sachets, or compressed into tablets. For example, the blend may be formulated in a dosage form at a strength of 750 mg levetiracetam.

### Example 6

### Extended-Release Composition with Hydrophilic Polymer

The following example illustrates an embodiment of an extended-release composition that incorporates hydrophilic polymers in the reservoir particulate.

TABLE 6
Composition

| S. No. | Ingredient | Quantity (% w/w) |
|---|---|---|
| 1 | Levetiracetam | 64 |
| 2 | Ethyl cellulose (from aqueous dispersion, | 15 |
| | quantity in solid content) | |
| 3 | Other solids in ethyl cellulose aqueous | 5 |
| | dispersion | |
| 4 | Hydroxypropyl methylcellulose | 13 |
| 5 | Water | q.s. |
| 6 | Magnesium Stearate | 3 |
| | Total | 100 |

The reservoir particulate was made as in Example 1 except that a release rate modifier was added to the aqueous ethyl cellulose dispersion. In this example the release rate modifier was a hydrophilic polymer sold under the trademark Methocel™ E5 Premium LV.

The aqueous ethyl cellulose dispersion with the release rate modifier was used to coat the Levetiracetam to produce a reservoir particulate as in Example 1. The reservoir particulate may be filled into capsules or sachets.

The dried reservoir particulate was lubricated with magnesium stearate. The final blend was then filled into capsules or sachets, or compressed into tablets. For example, the blend may be formulated in a dosage form at a strength of 750 mg levetiracetam.

### Example 7

### Extra-Particulate Release Rate Modifier

This example illustrates embodiments similar to that of Example 3 but with hydroxypropyl methylcellulose and magnesium stearate as the only extra-granular components in the formulation.

The reservoir particulate was produced as in Example 2. The reservoir particulate may be filled into capsules or sachets.

The reservoir particulate was subsequently blended with a release rate modifier, hydroxypropyl methylcellulose, then lubricated with magnesium stearate. The release rate modifier was a hydrophilic polymer sold under the trademark Methocel™ K100M Premium CR. The final blend was then filled into capsules or sachets, or compressed into tablets. For example, the blend may be formulated in a dosage form at a strength of 750 mg levetiracetam.

TABLE 7
Composition

| S. No. | Ingredient | Quantity (% w/w) |
|---|---|---|
| 1 | Levetiracetam | 72 |
| 2 | Ethyl cellulose (from aqueous dispersion, | 12 |
| | quantity in solid content) | |
| 3 | Other solids in ethyl cellulose aqueous | 4 |
| | dispersion | |
| 4 | Hydroxypropyl methylcellulose | 4 |
| | (in reservoir) | |
| 5 | Water | q.s. |
| 6 | Hydroxypropyl methylcellulose | 6 |
| | (extra-granular) | |
| 7 | Magnesium Stearate | 2 |
| | Total | 100 |

### Example 8

### Extra-Particulate Release Rate Modifier

This example illustrates embodiments similar to that of Example 3 but with hydroxypropyl methylcellulose and magnesium stearate as the only extra-granular components in the formulation.

The reservoir particulate was produced as in Example 2. The reservoir particulate may be filled into capsules or sachets.

The reservoir particulate was subsequently blended with a release rate modifier, hydroxypropyl methylcellulose, then lubricated with magnesium stearate. The release rate modifier was a hydrophilic polymer sold under the trademark Methocel™ K100M Premium CR. The final blend was then filled into capsules or sachets, or compressed into tablets. For example, the blend may be formulated in a dosage form at a strength of 500 mg levetiracetam.

TABLE 8
Composition

| S. No. | Ingredient | Quantity (% w/w) |
|---|---|---|
| 1 | Levetiracetam | 70 |
| 2 | Ethyl cellulose (from aqueous dispersion, | 12 |
| | quantity in solid content) | |
| 3 | Other solids in ethyl cellulose aqueous | 4 |
| | dispersion | |
| 4 | Hydroxypropyl methylcellulose | 3 |
| | (in reservoir) | |
| 5 | Water | q.s. |
| 6 | Hydroxypropyl methylcellulose | 8 |
| | (extra-granular) | |
| 7 | Magnesium Stearate | 3 |
| | Total | 100 |

### Example 9

### Extra-Particulate Release Rate Modifier

This example illustrates embodiments similar to that of Example 3 but with hydroxypropyl methylcellulose and magnesium stearate as the only extra-granular components in the formulation.

The reservoir particulate was produced as in Example 2. The reservoir particulate may be filled into capsules or sachets.

The reservoir particulate was subsequently blended with a release rate modifier, hydroxypropyl methylcellulose, then lubricated with magnesium stearate. The release rate modifier was a hydrophilic polymer sold under the trademark Methocel™ K100M Premium CR. The final blend was then filled into capsules or sachets, or compressed into tablets. For example, the blend may be formulated in a dosage form at a strength of 500 mg levetiracetam.

TABLE 9
Composition

| S. No. | Ingredient | Quantity (% w/w) |
|---|---|---|
| 1 | Levetiracetam | 68 |
| 2 | Ethyl cellulose (from aqueous dispersion, | 12 |
| | quantity in solid content) | |
| 3 | Other solids in ethyl cellulose aqueous | 4 |
| | dispersion | |
| 4 | Hydroxypropyl methylcellulose | 4 |
| | (in reservoir) | |
| 5 | Water | q.s. |
| 6 | Hydroxypropyl methylcellulose | 9 |
| | (extra-granular) | |
| 7 | Magnesium Stearate | 3 |
| | Total | 100 |

### Example 10

### Dissolution Profiles

This example illustrates that extended-release compositions described herein have dissolution profiles that allows for the administration of levetiracetam in a once-daily format.

The extended release tablets of Examples 1, 3, 4, 5, 6, 7, and 9 were tested for dissolution of levetiracetam from the dosage form in 900 mL of pH 6.0 phosphate buffer as the dissolution media maintained at 37° C. Basket method (USP Apparatus 1) at 100 rpm was employed, using the Basket dissolution method described in U.S. Pharmacopeia <711> Dissolution. Commercially available Keppra XR® (keppraxr.com) was used as a standard reference.

750 mg tablets were used for the formulations described in Tables 1, 3, 4, 5, 6, and 7, and 500 mg tablets were used for the formulations described in Tables 8 and 9.

The dissolution profiles are presented in Table 10 below.

TABLE 10
Dissolution Profile
Percentage Levetiracetam Dissolved

| Time | | | | | | | | | Keppra |
|---|---|---|---|---|---|---|---|---|---|
| (hours) | Example 1 | Example 3 | Example 4 | Example 5 | Example 6 | Example 7 | Example 8 | Example 9 | XR ® |
| 1 | 41 | 34 | 30 | 35 | 33 | 33 | 34 | 32 | 25 |
| 2 | 55 | 48 | 44 | 47 | 45 | 47 | 50 | 47 | 42 |
| 4 | 72 | 65 | 62 | 61 | 60 | 65 | 70 | 66 | 62 |
| 6 | 84 | 76 | 75 | — | — | 77 | 82 | — | 74 |
| 8 | 92 | 85 | 84 | 78 | 76 | 85 | 91 | 86 | 86 |
| 12 | 101 | 95 | 96 | 88 | 86 | 95 | 98 | 93 | 98 |

Table 10 shows that the formulations provided for herein have a biphasic release and a dissolution profile similar to and are comparable with Keppra XR®.

### Example 11

### In Vivo Bioavailability

This example illustrates the bioavailability of extended-release formulations provided for herein.

The extended release tablets of Example 4 (750 mg) were tested for bioavailability under fasting conditions and compared to Keppra XR® 750 mg tablets.

A 2-arm, open-label, single-dose, fasted relative bioavailability study of the levetiracetam extended release tablets consisting formulation compositions as shown in Example 4 versus Keppra XR® 750 mg tablet reference (“Reference”) was performed in 12 healthy, adult subjects. Each subject participated in two dosing periods separated by a washout period of 7 days. Dosing regimens were given after an overnight fast. Levetiracetam plasma concentrations in the blood samples were measured and compared.

The levetiracetam concentration-time data were used to calculate the following pharmacokinetic parameters: AUC₀₋ₜ, AUC_{0-∞}, Cₘₐₓ, Tₘₐₓ and t_{1/2}. The pharmacokinetic parameters were evaluated statistically by an analysis of variance (ANOVA). Analyses of AUC₀₋ₜ, AUC_{0-∞} and Cₘₐₓ were performed on Ln-transformed data.

The in vivo bioequivalence study results are summarized in Table 11.

TABLE 11
Formulation Example 4 versus Keppra XR ® 750 mg, Fasting, N = 12

| | Test Drug | Reference Drug | Ratio | Ln (Ratio | 90% C.I. |
|---|---|---|---|---|---|
| | Example 4 | Keppra XR ® | of T/R | of T/R) | (%) |
| AUC₀₋ₜ | 181 | 179 | 1.02 | 1.01 | (95.74, 107.19) |
| (μg · hr/mL) | | | | | |
| AUC_{0-∞} | 191 | 189 | 1.02 | 1.01 | (95.27, 107.42) |
| (μg · hr/mL) | | | | | |
| Cₘₐₓ | 10.3 | 10.1 | 1.03 | 1.01 | (91.53, 111.71) |
| (μg/mL) | | | | | |
| Tₘₐₓ (hr) | 4.17 | 4.17 | | | |
| t_{1/2} (hr) | 7.78 | 7.89 | | | |

Data in Table 11 indicate that the Test Drug (Example 4) is bioequivalent to the Reference Drug (Keppra XR®) under fasting conditions (within 90% confidence interval of 80-125%) for both AUC and Cₘₐₓ.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### CITATION LIST

### Patent Literature

US2006/0165796—UCB (now U.S. Pat. No. 7,858,122)
US2007/0298098—Elan
US2009/0099151—Panacea Biotec (see also WO 2007/000778)
US2009/0263481—Dr. Reddy's
US2010/0055177—Teva
US2010/0159009—Mutual
US2010/0151018-RD-Pharmagal (see also WO 2008/113901)
US2010/0310652 (see also WO 2009/087675)
US2011/0020445—Sanovel
US2011/0027359—UCB
US2011/0250282—UCB (see also WO 2010/057870)
WO 2006/123357—Sun
WO 2008/006528—UCB
WO 2009/069089—Ranbaxy
WO 2010/057869—UCB
WO 2011/159491—Mylan

### Non-Patent Literature

Iqbal et al., J. Chem. Soc. Pak. 33(5):634-639 (2011)

What is claimed is:

1. A reservoir particulate comprising a levetiracetam core coated with an aqueous dispersion that comprises at least one hydrophobic polymer, wherein the aqueous dispersion is free or substantially free of organic solvents.

2. The reservoir particulate of claim 1, wherein the levetiracetam core is levetiracetam, a pharmaceutically acceptable salt, solvate, hydrate, crystalline form, non-crystalline form or combination thereof.

3. The reservoir particulate of claim 1, wherein the levetiracetam is present at a concentration of about 50% w/w to about 95% w/w.

4. The reservoir particulate of claim 3, wherein the levetiracetam is present at a concentration of about 60% w/w to about 90% w/w.

5. The reservoir particulate of claim 4, wherein the levetiracetam is present at a concentration of about 73% w/w to about 79% w/w.

6. The reservoir particulate of claim 1, wherein the hydrophobic polymer is selected from ethyl cellulose, cellulose acetate, polyvinyl acetate, methacrylic acid esters neutral polymer, polyvinyl alcohol-maleic anhydride copolymers and combinations thereof.

7. The reservoir particulate of claim 6, wherein the hydrophobic polymer is ethyl cellulose.

8. The reservoir particulate of claim 1, wherein the hydrophobic polymer is present at a concentration of about 5% w/w to about 50% w/w.

9. The reservoir particulate of claim 8, wherein the hydrophobic polymer is present at a concentration of about 10% w/w to about 30% w/w.

10. The reservoir particulate of claim 9, wherein the hydrophobic polymer is present at a concentration of about 10% w/w to about 20% w/w

11. The reservoir particulate of claim 1, wherein the aqueous dispersion further comprises at least one hydrophilic polymer.

12. The reservoir particulate of claim 11, wherein the hydrophilic polymer is selected from copolyvidone, polyvinyl pyrrolidone, polyethylene glycols, hydroxyethyl cellulose, hydroxypropyl methylcellulose, hydroxypropyl cellulose and combinations thereof.

13. The reservoir particulate of claim 12, wherein the hydrophilic polymer is hydroxypropyl methylcellulose.

14. The reservoir particulate of claim 11, wherein the hydrophilic polymer is present at a concentration of about 0% to about 19% w/w.

15. The reservoir particulate of claim 14, wherein the hydrophilic polymer is present at a concentration of about 0% w/w to about 5% w/w.

16. The reservoir particulate of claim 15, wherein the hydrophilic polymer is present at a concentration of about 5% w/w to about 15% w/w.

17. The reservoir particulate of claim 11, wherein the levetiracetam concentration is about 50% w/w to about 95% w/w, the hydrophobic polymer concentration is about 5% w/w to about 50% w/w, and the hydrophilic polymer at a concentration is about 0.1% to about 19% w/w.

18. The reservoir particulate of claim 11, wherein the hydrophobic polymer is ethyl cellulose and the hydrophilic polymer is hydroxypropyl methylcellulose.

19. The reservoir particulate of claim 1, wherein the reservoir particulate comprises particles.

20. The reservoir particulate of claim 1, wherein the reservoir particulate comprises agglomerates.

21. The reservoir particulate of claim 1, wherein the hydrophobic polymer, when compressed into tablet dosage form, forms a crosslinked structure (matrix) which will not dissolve in water.

22. The reservoir particulate of claim 21, wherein the hydrophobic polymer maintains the original shape of the dosage form for at least about 12 hours in vitro.

23. The reservoir particulate of claim 1, wherein the hydrophobic polymer provides a mechanism controlling levetiracetam release from the reservoir particulate.

24. A dosage form comprising the reservoir particulate of claim 1.

25. The dosage form of claim 24, comprising a tablet, a capsule, a sachet, mini-tabs, pellets, or free flowing granules.

26. A reservoir particulate according to claim 1, wherein the aqueous dispersion is coated onto the levetiracetam during granulation.

27. A reservoir particulate according to claim 26, wherein the aqueous dispersion further comprises at least one hydrophilic polymer.

28. The reservoir particulate of claim 27, wherein the hydrophilic polymer is mixed with the hydrophobic polymer prior to addition to the levetiracetam.

29. The reservoir particulate of claim 28, wherein the aqueous dispersion further comprises at least one excipient selected from a plasticizer, a suspending agent, an anti-caking agent, an emulsifying agent, a stabilizing agent and an anti-coagulation agent.

30. The reservoir particulate of claim 28, wherein the hydrophobic polymer and the hydrophilic polymer form a coating on the levetiracetam.

31. The reservoir particulate of claim 30, wherein the coating is partial.

32. A method of manufacturing an extended-release pharmaceutical composition, said method comprising:
(a) coating levetiracetam with an aqueous dispersion that comprises at least one hydrophobic polymer to form a reservoir particulate, wherein the aqueous dispersion is free of or substantially free of organic solvents;
(b) blending the reservoir particulates of step (a) with one or more pharmaceutically acceptable excipient to form a dry blend;
(c) forming a final dosage form from the dry blend.

33. The method of claim 32, wherein the reservoir particulate comprise a levetiracetam core that comprises levetiracetam, a pharmaceutically acceptable salt, solvate, hydrate, crystalline form, non-crystalline form or combination thereof.

34. The method of claim 32, wherein the coating is done in a spray dry granulator, a fluid bed granulator, or a high shear granulator.

35. The method of claim 32, wherein the hydrophobic polymer is ethyl cellulose.

36. The method of claim 32, wherein the pharmaceutically acceptable excipient is selected from hydrophilic polymers, binders, lubricants, glidants, disintegrants, fillers, diluents and combinations thereof.

37. The method of claim 32, wherein the aqueous dispersion further comprises at least one hydrophilic polymer.

38. The method of claim 37, wherein the hydrophilic polymer is hydroxypropyl methylcellulose.

39. The method of claim 32, further comprising sizing the reservoir particulate of step (a)

40. The method of claim 38, wherein the reservoir particle has a particle size of about 10 μm to about 1000 μm.

41. The method of claim 40, wherein the moisture content of the reservoir particles is about 0.05% w/w to about 5% w/w measured by weight loss on drying (LOD) at 105° C.

42. The method of claim 32, wherein the final dosage form comprises a tablet, a capsule, a sachet, mini-tabs, pellets, or free flowing granules.

43. An extended-release pharmaceutical composition prepared by the method of claim 32.

44. An extended-release pharmaceutical composition prepared by the method of claim 37.

45. An extended-release pharmaceutical composition comprising at least one reservoir particulate of claim 1, wherein the composition is substantially free or free of organic solvents.

46. The extended-release pharmaceutical composition of claim 45, wherein the reservoir particulate comprises a levetiracetam core that comprises levetiracetam, a pharmaceutically acceptable salt, solvate, hydrate, crystalline form, non-crystalline form or combination thereof.

47. The extended-release composition of claim 45, wherein the hydrophobic polymer is ethyl cellulose.

48. The extended-release pharmaceutical composition of claim 45, wherein the aqueous dispersion further comprises at least one hydrophilic polymer.

49. The extended-release pharmaceutical composition of claim 48, wherein the hydrophilic polymer is hydroxypropyl methylcellulose.

50. The extended-release pharmaceutical composition of claim 45, further comprising an extra-particulate matrix, wherein said matrix comprises at least one pharmaceutically acceptable excipient.

51. The extended-release pharmaceutical composition of claim 50, wherein the extra-particulate matrix comprises at least one hydrophilic polymer.

52. The extended-release pharmaceutical composition of claim 51, wherein the hydrophilic polymer is hydroxypropyl methylcellulose.

53. The extended-release pharmaceutical composition of claim 50, wherein both the reservoir particulate and the extra-particulate matrix comprise at least one hydrophilic polymer, wherein the hydrophilic polymer(s) in the reservoir particulate and the extra-particulate matrix are the same or different.

54. The extended-release composition of claim 53, wherein the hydrophilic polymer in both the reservoir particulate and the extra-particulate matrix is hydroxypropyl methylcellulose.

55. The extended-release pharmaceutical composition of claim 50, comprising a matrix comprising at least one hydrophilic material, one glidant, one diluent and/or one lubricant, and at least one reservoir particulate comprising levetiracetam or a derivative thereof coated with at least one hydrophobic polymer, one hydrophilic polymer, or a combination thereof in an aqueous dispersion, wherein the composition is substantially free or free of organic solvents.

56. The extended-release pharmaceutical composition of claim 45, further comprising a controlled release layer.

57. The extended-release composition of claim 56, wherein the controlled release layer comprises at least one hydrophobic excipient.

58. The extended-release pharmaceutical composition of claim 56, wherein the controlled release layer comprises at least one hydrophilic excipient.

59. The extended-release pharmaceutical composition of claim 45, wherein the composition is produced without added organic solvents.

60. The extended-release pharmaceutical composition of claim 45, wherein said pharmaceutical composition is intended to be administered once daily.

61. The extended-release pharmaceutical composition of claim 45, wherein the levetiracetam is present at a concentration of about 30% w/w to about 95% w/w.

62. The extended-release pharmaceutical composition of claim 61, wherein the levetiracetam is present at a concentration of about 50% w/w to about 90% w/w.

63. The extended-release pharmaceutical composition of claim 45, wherein the hydrophobic polymer is present at a concentration of about 2% w/w to 50% w/w.

64. The extended-release pharmaceutical composition of claim 63, wherein the hydrophobic polymer is present at a concentration of about 5% w/w to about 30% w/w.

64. The extended-release pharmaceutical composition according to claim 47, further comprising at least one hydrophilic polymer at a concentration of about 5% w/w to about 15% w/w.

65. The extended-release pharmaceutical composition of claim 45, wherein the pharmaceutical composition releases the levetiracetam contained therein over a period of about 12 hours after introduction of the dosage form into the dissolution medium when tested in 900 mL of pH 6.0 phosphate buffer maintained at 37° C. using a basket method (USP Apparatus 1) at 100 rpm.

66. The extended-release pharmaceutical composition of claim 65, wherein after twelve hours about 85 wt. % to about 100 wt. % of the total amount of the active agent is released.

67. The extended-release pharmaceutical composition of claim 45, wherein the extended-release composition is bioequivalent to a reference drug with a proprietary name of Keppra XR® when administered to a patient in a fasted or non-fasted state.

68. The extended-release pharmaceutical composition of claim 45, wherein the levetiracetam is present in an amount of about 250 mg to about 1500 mg.

69. The extended-release pharmaceutical composition of claim 68, wherein the levetiracetam is present in an amount of about 500 mg.

70. The extended-release pharmaceutical composition of claim 68, wherein the levetiracetam is present in an amount of about 750 mg.

71. The extended-release pharmaceutical composition of claim 55, wherein the reservoir particulate comprises a plurality of reservoir particulates.

72. The extended-release pharmaceutical composition of claim 48, wherein the hydrophobic polymer is ethyl cellulose and the hydrophilic polymer is hydroxypropyl methylcellulose.
